(19) United States
(12) Patent Application Publication
(10) Pub. No.: US 2011/0076709 A1
(43) Pub. Date: Mar. 31, 2011

(54) Method for the Diagnosis of Leukemia Using Caspase-3

(75) Inventors: Jean-Marie Bruey, Encinitas, CA (US); Maher Albitar, Coto De Caza, CA (US)

(73) Assignee: Quest Diagnostics Investments Incorporated

(21) Appl. No.: 12/887,435

(22) Filed: Sep. 21, 2010

Related U.S. Application Data
(60) Provisional application No. 61/266,915, filed on Dec. 4, 2009.
(60) Provisional application No. 61/246,486, filed on Sep. 28, 2009.

(51) Int. Cl. C12Q 1/37

(52) U.S. Cl. 435/23

(57) ABSTRACT

The invention provides methods for determining the prognosis of a patient diagnosed with a leukemia, including B-cell chronic lymphocytic leukemia and acute myelogenous leukemia, by measuring the level of caspase-3 activity in a biological sample. The invention also relates to the diagnosis of leukemia, including B-cell chronic lymphocytic leukemia and acute myelogenous leukemia.

### CROSS-REFERENCE TO RELATED APPLICATION

This application claims the benefit of U.S. provisional applications 61/246,486, filed Sep. 28, 2009, and 61/266,915, filed Dec. 4, 2009, each of which is hereby incorporated by reference in its entirety.

### TECHNICAL FIELD

The present invention relates generally to the field of cancer diagnostics and, in particular, the diagnosis and prognosis of patients having leukemia.

### BACKGROUND

The following description is provided to assist the understanding of the reader. None of the information provided or references cited is admitted to be prior art to the present invention.

Cancer describes a class of disorders and diseases characterized by the uncontrolled growth of aberrant cells. Currently, cancer is one of the most deadly diseases with about 1.2 million new cases of cancer being diagnosed each year in the United States of America alone.

One form of cancer, accounting for about 3% of all cancers in the United States of America, is leukemia. This malignant disease is characterized by an abnormal proliferation of white blood cells which can be detected in the peripheral blood and/or bone marrow. Leukemia can be broadly classified into acute and chronic leukemia. Further classification within these groups is essential as a precise diagnosis is necessary in order to determine prognosis and guide the choice of treatment. The acute leukemias can be subclassified into myeloid and lymphoid leukemias in a variety of ways, including cell morphology and cytochemistry. Despite the obvious morphological differences between the myeloid and lymphoid leukemias, immunophenotyping and cytogenetic analysis have become increasingly important in recent years to confirm and/or supplement diagnosis of leukemias.

Acute myeloid leukemia (AML) is the most common form of leukemia accounting for about 50% of all leukemia cases and even 85% of all acute leukemia cases involving adults. As a general criterion for the classification as AML, the cytoplasmic antigen myeloperoxidase should be demonstrated in the abnormal cell population either cytochemically or immunologically. Furthermore, the diagnosis of AML is supported by the expression of lineage-associated markers. Only a limited number of markers commonly expressed by a large number of AML-subtypes, such as CD13 and CD33, have been identified.

B-cell chronic lymphocytic leukemia (B-CLL) is characterized by a progressive accumulation of long-lived and well-differentiated clonal B-lymphocytes. Although B-CLL pathogenesis is not entirely understood, the progressive increase in lymphocyte counts coupled with the very low proportion of proliferating cells suggests that B-CLL may be primarily driven by defective apoptosis. B-CLL has a highly varied course in affected subjects. B-CLL can be categorized in at least two different subgroups, with different clinical outcome. See, e.g. Wiestner, A., Rosenwald, A., Barry, T. S., Wright, G., et al., Blood, 101:4944-4951 (2003); Hamblin, T., Ann Hematol 2002, 81: 99-303 (2002); Rozman, C. and Montserrat, E., N Engl J Med, 333:1052-1057 (1995); Chen, L., Widhopf, G., Huynh, L., Rassenti, L., et al., Blood, 100:4609-4614 (2002).

Some patients have an indolent disease with little need for therapeutic intervention, while other patients show a more aggressive clinical course requiring therapeutic intervention. Less aggressive forms of B-CLL are generally monitored but not treated since the risks associated with therapy can outweigh the benefits. However, other forms of the disease can progress rapidly resulting in uncomfortable symptoms, repeated serious infections and death, warranting aggressive therapeutic intervention such as bone marrow transplant, chemotherapy and monoclonal antibody therapy. The significant differences in clinical outcome associated with diverse forms of B-CLL make it especially important to be able to distinguish more aggressive forms of the disease from less aggressive forms.

Several markers have been identified which may have prognostic value in B-CLL including beta-2-microglobulin (B2M), immunoglobulin variable region heavy chain gene (IgV_{H}) mutational status, ZAP-70, CD-38, lipoprotein lipase and certain chromosomal abnormalities such as 11q, 13q and 17p deletions. See, e.g., Damle. R. N., Wasil, T., Fais, F., Ghiotto, F., et al., Blood, 94:1840-1847 (1999); Hamblin, T. J., Davis, Gardiner, A., Oscier, D. G. and Stevenson, F. K., Blood, 94:1848-1854 (1999); Rassenti, L. Z., Huynh, L., Toy, T. L., Chen, L., et al., N Eng J Med, 351:893-901 (2004); Krober, A., Seiler, T., Benner, A., Bullinger, L., et al., Blood, 100:1410-1416 (2002); Oscier, D. G., Gardiner, A. C., Mould, S. J., Glide, S., et al., Blood, 100:1177-1184 (2002); Rassenti, L. Z., Huynh, L., Toy, T. L., Chen, L., et al., N Engl J Med, 351:893-901 (2004); Orchard, J. A., Ibbotson, R. E., Davis, Z., Wiestner, A., et al., Lancet, 363:105-111 (2004); Wiestner, A., Rosenwald, A., Barry, T. S., Wright, G., et al., Blood, 101:4944-4951 (2003). However, no single marker appears to be definitive in classifying B-CLL patients and it is possible that clonal evolution of cells involved in B-CLL results in heterogeneous populations of cells which change over time.

### SUMMARY OF THE INVENTION

This invention relates to detection of caspase-3 in a biological sample. The invention is useful for detecting the level of caspase-3 in such samples for prognosis and diagnosis relating to leukemia, e.g., B-CLL and AML.

In one aspect, the invention provides a method of determining a prognosis for a subject diagnosed with a leukemia comprising: i) measuring the level of caspase-3 present in a biological sample from said subject; ii) measuring the number of circulating lymphocytes in a biological sample of peripheral blood from said subject; iii) calculating a caspase-3 index by normalizing the measured level of caspase-3 to the number of circulating lymphocytes; iv) diagnosing the subject as having a poor prognosis wherein the caspase-3 index is greater than a diagnostic cut-off value.

In another aspect, the invention provides a method for diagnosing a leukemia in a subject comprising: i) measuring the level of caspase-3 present in a biological sample from a subject; ii) comparing the level of caspase-3 to a diagnostic cut-off value; and iii) diagnosing the subject as having a leukemia wherein the absolute level of caspase-3 from the biological sample is lower than the absolute level of caspase-3 identified by the diagnostic cut-off value.

In one embodiment of all aspects of the invention, the biological sample is a body fluid, including acellular body fluids such as, for example, blood serum or plasma.

In another embodiment of all aspects of the invention, the leukemia is chronic lymphocytic leukemia, acute lymphoblastic leukemia, acute myelogenous leukemia, chronic myelogenous leukemia, hairy cell leukemia, T-cell prolymphocytic leukemia, large granular lymphocytic leukemia, B-cell chronic lymphocytic leukemia, or myelodysplastic syndrome.

In any of the aspects of the invention, the level of caspase-3 may be the relative or absolute amount of caspase-3 protein or the measurable amount of caspase-3 enzymatic activity (“caspase-3 activity”). In certain embodiments of all aspects of the invention, the caspase-3 is measured by any assay, In some embodiments, the caspase-3 activity is measured by an assay comprising a caspase-3 peptide substrate. In some embodiments, the caspase-3 peptide substrate is linked to a detectable marker. Suitable detectable markers include, but are not limited to, a colorimetric moiety, a fluorescent moiety or a radioactive moiety. In some embodiments, the caspase-3 substrate comprises the peptide DEVD.

In certain embodiments of all aspects of the invention, the caspase-3 level in the control sample is measured prior to measuring the level of caspase-3 in the biological sample. In some embodiments, the caspase-3 level present in the control sample is a pre-determined reference level which was measured from a reference sample (e.g., the level of caspase-3, as measured in a sample, taken previously and/or measured previously from the same or different individual or individuals). The reference sample may have been taken from the same individual as the subject of the method of the invention at an earlier time point. Alternatively, the reference sample may be from an individual or individuals that are different from the subject of the method of the invention.

In certain embodiments of all aspects of the invention, the caspase-3 level and/or the caspase 3 index measured in a sample obtained from an individual is compared to a cut-off value in order to make a diagnostic or prognostic assessment. In some embodiments, the cut-off value for the caspase-3 index is at least 5 pmol/min/1000 lymphocytes, at least 6 pmol/min/1000 lymphocytes, at least 7 pmol/min/1000 lymphocytes, at least 8 pmol/min/1000 lymphocytes, at least 9 pmol/min/1000 lymphocytes, at least 10 pmol/min/1000 lymphocytes, at least 11 pmol/min/1000 lymphocytes, at least 12 pmol/min/1000 lymphocytes, at least 13 pmol/min/1000 lymphocytes, at least 14 pmol/min/1000 lymphocytes, or at least 15 pmol/min/1000 lymphocytes. In other embodiments, the cut-off value for caspase-3 enzymatic activity is about 5.0, 5.5, 6.0, 6.5, 7.0, or 7.5 pmol/min/μl.

### BRIEF DESCRIPTION OF THE FIGURES

FIG. 1 is a graph depicting the survival rate of patients with B-CLL over a time period of approximately 66 months. Patients were separated into two groups: 1) patients which had a normalized caspase-3 activity equal to or less than 8 pmol/min/1000 lymphocytes (solid line) and 2) patients which had a normalized caspase-3 activity greater than 8 pmol/min/1000 lymphocytes (dotted line).

FIG. 2 is a box plot of the levels of circulating caspase-3 activity in plasma in Normal individuals (n=98) and AML patients (n=108) as determined by a caspase-3 fluorimetric assay. The units of caspase-3 activity are expressed as pmol/min. Median values of the levels of caspase-3 activity is represented as a small square within the box in each case.

FIG. 3 is a Kaplan-Meier curve depicting the cumulative proportion of surviving patients with AML (as a percentage of total patients) over a time period of approximately 78 months. Patients were separated into two groups: 1) patients which had a normalized caspase-3 activity equal to or less than 14 pmol/min/1000 lymphocytes (solid line) and 2) patients which had a normalized caspase-3 activity greater than 14 pmol/min/1000 lymphocytes (dotted line).

### DETAILED DESCRIPTION OF THE INVENTION

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a nucleic acid” includes a combination of two or more nucleic acids, and the like.

The term “diagnose” or “diagnosis” or “diagnosing” as used herein refer to distinguishing or identifying a disease, syndrome or condition or distinguishing or identifying a person having a particular disease, syndrome or condition. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. In this regard, the term means assessing whether or not an individual or a subject has a level of caspase activity consistent with having a leukemia, e.g., B-CLL.

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of years, on average that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The term “poor prognosis” as used herein, in the context of a patient having a leukemia, e.g. B-CLL, refers to an increased likelihood that the patient will have a worse outcome in a clinical condition relative to a patient diagnosed as having the same disease but having a caspase-3 index which is greater than a specified cut-off value or a caspase-3 level which is lower than a specified cut-off value. A poor prognosis may be expressed in any relevant prognostic terms and may include, for example, the expectation of a reduced duration of remission, reduced survival rate, and reduced survival duration.

“Caspase-3 index,” as used herein, refers to a normalization of the absolute value of measured caspase-3 activity from a biological sample. The absolute caspase-3 activity may be normalized to any convenient or clinically important measure (e.g., amount of serum protein, hemoglobin, etc.). In some embodiments, the absolute caspasc-3 activity is normalized to the number of circulating lymphocytes in peripheral blood.

A “cut-off value,” as used herein, refers to a diagnostic or prognostic criterion against which a patient value is compared. For example, the caspase-3 index calculated for a particular individual is compared to a predetermined cut-off value in order to arrive at a diagnosis (i.e., diseased or health). Such a comparison may be the sole basis for make a particular diagnosis or prognosis, or the comparison may be used in conjunction with other factors such as clinical and demographic factors and/or the level of other markers of disease. A cut-off value may be determined by any medically appropriate means. For example, a cut-off value may be the average (mean or medium) of a particular measure in a population of individuals known to have disease or be disease-free. Alternatively, the cut-off value may represent the value which defines an extreme percentile in that population (e.g., the value which correctly identifies 95%, 99%, 99.5%, 99.9%, etc of individuals) which reduces the number of false positives or false negatives to a medically acceptable level. Alternatively, the cut-off value may be the value determined in a single individual.

As used herein, the term “sample,” “biological sample” refers to any liquid or solid material obtained from a biological source, such a cell or tissue sample or bodily fluids. “Bodily fluids” may include, but are not limited to, blood, serum, plasma, saliva, cerebrospinal fluid, pleural fluid, tears, lactal duct fluid, lymph, sputum, urine, saliva, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin. Exemplary sample tissues include, but are not limited to bone marrow or tissue (e.g. biopsy material).

“Plasma,” as used herein, refers to acellular fluid found in blood. “Plasma” may be obtained from blood by removing whole cellular material from blood by methods known in the art (e.g., centrifugation, filtration, and the like). As used herein, “peripheral blood plasma” refers to plasma obtained from peripheral blood samples.

“Serum,” as used herein, includes the fraction of plasma obtained after plasma or blood is permitted to clot and the clotted fraction is removed.

“Lymphocytes,” as used herein, refer to cells of the immune system which are a type of while blood cell. Lymphocytes include, but are not limited to, T-cells (cytotoxic and helper T-cells), B-cells and natural killer cells (NK cells)

“Subject” or “individual” or “patient,” as used herein, refers to one who receives medical care, attention or treatment and/or for whom diagnosis, detection, prognosis or therapy is desired. Mammalian patients include but are not limited to humans, domestic animals, farm animals, and zoo, sport, or pet animals such as dogs, cats, horses, cattle, swine and other such animals which may contract leukemias. As used herein, the term is meant to encompass an individual diagnosed with a disease such as leukemia as well as a person who may be symptomatic for a disease but who has not yet been diagnosed.

“Assay” or “assaying” as used herein means qualitative or quantitative analysis or testing.

“Detectable label” as used herein refers to a molecule or a compound or a group of molecules or a group of compounds used to measure caspase-3 activity. In some cases, the detectable label may be detected directly. In other cases, the detectable label may be a part of a binding pair, which can then be subsequently detected. Signals from the detectable label may be detected by various means and will depend on the nature of the detectable label. Detectable labels may be isotopes, fluorescent moieties, colored substances (i.e. colorimetric moieties), and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

“About” as used herein means in quantitative terms, plus or minus 10%.

The present invention provides methods for diagnosis of leukemia and for determining a prognosis for a subject diagnosed with a leukemia by detecting and measuring the level of caspase-3 activity in a biological sample.

### Caspase-3

Caspase-3 is also known as CPP32, Yama and Apopain. It is responsible for partial or total proteolytic cleavage of many key proteins. See Cohen, “Caspases: the executioners of apoptosis,” J. of Biochem. 326:1-16 (1997). Caspase-3 is a member of the CED-3 family of caspases and is expressed widely in lymphocytic cell lines, suggesting that it is an important mediator of apoptosis in the immune system. See id.

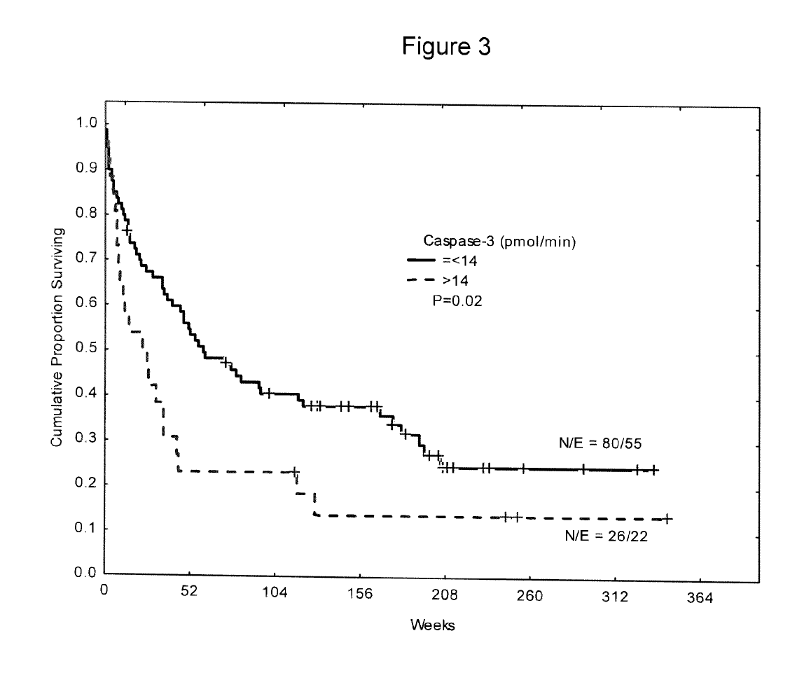

Caspase-3, like all caspases, are synthesized as inactive proenzymes with an N-terminal prodomain. Id. at 6. Caspases are cleaved at specific aspartate cleavage sites, resulting in their activation. Id. at 2. Caspase-3 may be cleaved at the aspartate residue at position 9 (Asp-9) of the full-length procaspase or at the aspartate residue at position 28 (Asp-28). Id. at FIG. 3.

Caspase-3 recognizes and cleaves proteins with the following motif: DXXD. Caspase-3 cleaves after the last asparagine residue in the DXXD motif. Thus, peptides such as DEVD were developed as model substrates for caspase-3. However, any quaternary peptide (i.e. a 4-mer or tetramer) following the DXXD motif may be used as a substrate for caspase-3.

It is believed that the progressive increase in lymphocyte counts coupled with the very low proportion of proliferating cells suggests that B-CLL may be primarily driven by defective apoptosis. Since the proapoptotic enzyme, caspase-3 is activated at a point of convergence for the intrinsic and extrinsic apoptosis induction pathways, its activity gives a reliable measure of ongoing levels of apoptosis and thus a good diagnostic indicator of the presence and prognosis of a leukemia disease state, such as B-CLL.

### Sample

Biological samples may be of human or non-human origin. In one embodiment, the sample may be obtained from an individual who is suspected of having a disease, such as leukemia. In another embodiment, a biological sample may be obtained from a healthy individual who is assumed of having no disease, such as leukemia. In preferred embodiments, the sample may be obtained from leukemia patients. In some embodiments, the sample may be obtained from an individual diagnosed with leukemia. In other embodiments, the sample may be obtained from an individual diagnosed with B-cell neoplasms such as B-cell chronic lymphocytic leukemia (B-CLL).

In certain embodiments, an individual's plasma may be used as the biological sample from which caspase-3 activity in measured in the methods of the present invention. In another embodiment, an individual's serum may be used as the biological sample from which caspase-3 activity is measured in the methods of the present invention.

### Sample Collection and Preparation

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70 degrees centigrade until thawed and used. “Fresh” plasma or serum should be refrigerated or maintained on ice until used. Exemplary methods are described below.

### Detectable Label

Any detectable label may be used in assay to detect and measure caspase-3 activity. The detectable labels for use in the methods of the present invention may be conjugated to a caspase-3 peptide substrate for measuring caspase-3 activity.

Detectable labels include but are not limited to fluorophores, isotopes (e.g. 32P, 33P, 35S, 3H, 14C, 125I, 1311), electron-dense reagents (e.g., gold, silver), nanoparticles, enzymes commonly used in an ELISA (e.g., horseradish peroxidase, beta-galactosidase, luciferase, alkaline phosphatase), chemiluminiscent compound, colorimetric labels (e.g., colloidal gold), magnetic labels (e.g., Dynabeads™), biotin, digoxigenin, haptens, proteins for which antisera or monoclonal antibodies are available, ligands, hormones, oligonucleotides capable of forming a complex with the corresponding oligonucleotide complement.

In some embodiments, the detectable label is a fluorophore. Suitable fluorescent moieties include but are not limited to the following fluorophores working individually or in combination:

4-acetamido-4′-isothiocyanatostilbene-2,2′ disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalenc-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes; BODIPY® R-6G, BOPIPY® 530/550, BODIPY® FL; Brilliant Yellow; coumarin and derivatives: coumarin, 7-amino-4-methylcoumarin (AMC, Coumarin 120), and 7-amino-4-trifluoromethylcouluarin (Coumarin 151, AFC); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC(XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (TEXAS RED®); terbium chelate derivatives; N,N,N′,N′-tetramethyl-6-carboxyrhodamine (TAMRA); tetramethyl rhodamine; and tetramethyl rhodamine isothiocyanate (TRITC).

### Assay

There are a variety of assay formats known to those of ordinary skill in the art to measure the level of caspase-3 in a sample. Caspase-3 levels in the methods of the present invention may be measured by any means known to one of ordinary skill in the art.

The level of caspase-3 in a sample may be measured by absolute or relative protein levels, or concentration, of caspase-3 present in the sample. Additionally, the level of caspase-3 in a sample may be quantified by measuring the level of caspase-3 enzymatic activity present in a sample. Assays used for determining the amount of caspase-3 protein present in a sample may measure the total concentration of caspase-3 and procaspase-3 present in a sample or the assay may measure the concentration of caspasc-3 only, to the exclusion of procaspase-3.

Protein levels or concentration of caspase-3 may be measured by a variety protein quantification methods which are well known in the art. Non-limiting examples include, the use of directly or indirectly labelled polyclonal or monoclonal antibodies specifically directed to caspase-3. Caspase-3 antibodies include antibodies which bind caspase-3 and procaspase-3 as well as antibodies which only bind caspase-3 and not procaspase-3.

Depending on the biological sample, labelled caspase-3 protein could be detected directly on cells either by cytometric techniques (cell-shorter techniques, cellular suspensions, etc.) or by histochemical techniques (fixed cells, solid or semi-solid tissues). Cells could be previously permeabilized to allow the introduction of the appropriate caspase-3 antibody. Also labelled caspase-3 protein may be extracted and/or isolated from the biological sample and analyzed by Western blot techniques, after migration of the cell extracts on Polyacrylamide gels (PAGE-SDS). The quantitative intensities of the detected caspase-3 protein can then be compared on a Coomassie-stained SDS-PAGE. Alternatively, the specific band(s) of interest (e.g., band corresponding to caspase-3) can be detected by, for example, by immunoblot gel analysis and quantitative intensities detected by scanning densitometer. Caspase-3 isolated from a sample may also be quantified by standard protein quantification assays such as Lowry, Biuret, Bradford and BCA, which are all well-known in the art.

Additionally, the caspase-3 level present in a sample may be measured based on the enzymatic activity of the caspase-3 molecules present.

A non-limiting example of an assay to measure caspase-3 activity relies upon the use of a synthetic substrate peptide. One such peptide includes the amino acid sequence DEVD. Other peptides which may be used in such enzymatic assays include peptides containing the following motif DXXD. The synthetic caspase-3 peptide substrate may be linked to a detectable marker. The detectable marker may be linked on the C-terminal asparagine (or aspartic acid) residue. Caspase-3 cleaves the tetrapeptide DEVD between the C-terminal asparagine and the detectable marker. Once the marker has been separated from the peptide substrate the detectable marker may be quantified by a number of different methods.

Any detectable marker may be linked to the synthetic caspase-3 substrate. Non limiting examples include, fluorophores, chromophores, and radioactive isotopes as described previously. For example, the fluorophore 7-amino-4-trifluoromethylcoumarin (AFC) may be used. The conjugated substrate DEVD-AFC, emits a blue light (λₘₐₓ=400 nm). However upon cleavage of the substrate by caspase-3, the free AFC emits a blue-green light at 505 nm, which may be used to measure caspase-3 activity. As another example, the fluorophore 7-amino-4-methylcoumarin (AMC) may be used. Upon cleavage, AMC fluorescence can be measured using a 380-nm excitation filter and a 460-nm emission filter. Similarly, chromophores, such as p-nitroanilide (pNA), may be used which once cleaved can be measured by spectrophotometric detection.

Non-limiting examples of caspase-3 assays for use in measuring caspase-3 enzymatic activity include for example those assays sold commercially under the following names: ApoAlert® Caspase-3 (Clontech Laboratories, Inc); Caspase-3 Assay Kit (BD Biosciences); Caspase-3 Assay Kit (Sigma-Aldrich).

To determine the prognosis of a subject diagnosed with leukemia, e.g. B-CLL, the caspase-3 level in the biological sample (e.g., plasma) may be normalized to number of circulating lymphocytes as measured in the peripheral blood from the same patient. This value, which is referred to herein as the caspase-3 index is then compared to a predetermined cut-off value. The absolute level of caspase-3 (e.g., as measured by protein concentration or enzymatic activity) may also be compared to a predetermined cut-off value.

The cut-off value may be equivalent to the average mean caspase-3 index or caspase-3 level obtained from patients which have been diagnosed with a leukemia but have survived for a prolonged period of time. For example, a less aggressive form of a leukemia in which the patient has survived for more than about 5 years.

Generally, the cut-off value for the caspase-3 index is at least 5 pmol/min/1000 lymphocytes, at least 6 pmol/min/1000 lymphocytes, at least 7 pmol/min/1000 lymphocytes, at least 8 pmol/min/1000 lymphocytes, at least 9 pmol/min/1000 lymphocytes, at least 10 pmol/min/1000 lymphocytes, at least 11 pmol/min/1000 lymphocytes, at least 12 pmol/min/1000 lymphocytes, at least 13 pmol/min/1000 lymphocytes, at least 14 pmol/min/1000 lymphocytes, or at least 15 pmol/min/1000 lymphocytes. Thus, patients having a caspase-3 index greater than the cut-off value, will be considered to have a poor prognosis in the progression of the leukemia.

The cut-off value for caspase-3 enzymatic activity is about 5, 5.5, 6, 6.5, 7 or 7.5 pmol/min/μl. Patients having an absolute enzymatic value lower than the cut-off value may be diagnosed an aggressive form of the leukemia.

In an alternate method, the cut-off value is determined using a Receiver Operator Curve, according to the method of Sackett et al., Clinical Epidemiology: A Basic Science for Clinical Medicine, Little Brown and Co., 1985, pp. 106-107. Briefly, in this method, the cut-off value may be determined from a plot of pairs of true positive rates (i.e., sensitivity) and false positive rates (100%-specificity) that correspond to each possible cut-off value for the diagnostic result. The cut-off value on the plot that is the closest to the upper left-hand corner (i.e., the value that encloses the largest area) is the most accurate cut-off value, and a sample generating a signal that is higher than the cut-off value determined by this method may be considered to be from a patient with a poor prognosis. Alternatively, the cut-off value may be shifted to the left along the plot, to minimize the false positive rate, or to the right, to minimize the false negative rate. In general, a biological sample generating a signal that is higher than the cut-off value determined by these or any other methods known to one of skill in the art is considered to indicate a poor prognosis for the patient.

### Example 1

### Caspase-3 Activity in B-CLL Patients

### Sample Collection

Blood was collected in EDTA-containing tubes (Becton Dickinson, N.J.) from 194 individuals with B-CLL and 95 individuals without lymphoid malignancies. Plasma was separated from blood cells by centrifugation at 1000×g for 15 min. White blood cells were separated from RBC by lysis and centrifugation using Puregene® RBC lysis solution according to manufacturer's instructions (Qiagen, Valencia Calif.). The cell pellet was washed with phosphate-buffered saline. Both plasma and cell samples were cryopreserved at −80° C. for future use.

### Caspase-3 Assay

Caspase-3 activity was measured in each sample using an assay based on the caspase-3 peptide substrate DEVD. Five microliter of plasma was mixed with 50 μl of 2× reaction buffer with DTT. 5 μl of 1 mM of the DEVD conjugated substrate, DEVD-AFC (7-amino-4-trifluoromethylcoumarin), was added to yield a final concentration of 50 μM. The mixture was incubated at 37° C. for 1 hour in a water bath. Control reactions using the caspase-3 inhibitor (DEVD-CHO) or no caspase-3 substrate were performed in parallel. Samples were read in a fluorometer with a 400-nm excitation filter and a 505-nm emission filter in 96-well plates.

### Absolute Caspase-3 Activity

Circulating caspase-3 activity was easily detectable in the plasma of B-CLL patients and normal controls (i.e. patients without lymphoid malignancies). The levels of absolute caspase-3 activity were significantly lower (p=0.005) in B-CLL patients (median=7.49 pmol/min/μl, range 4.2-19.68 pmol/min/μl) as compared to control patients (median 8.27 pmol/min/μl, range 4.54-34.30 pmol/min/min). However, absolute levels of caspase-3 in plasma from patients with B-CLL did not correlate with any of the laboratory parameters, Raj stage or performance status.

### Normalized Caspase-3 Activity

In order to measure the relative apoptosis occurring in leukemic cells, the caspase-3 activity, as measured from plasma, was normalized to the number of circulating lymphocytes in peripheral blood to calculate a caspase-3 index.

Lymphocytes counts were determined from the routine white blood cell count and percent of lymphocyte in the routine blood count (complete blood cell count).

The caspase-3 index correlated negatively with hone marrow cellularity (p<0.001), spleen size (p=0.002), and number of sites of enlarged lymph nodes (p<0.001). Interestingly, there was positive correlation between the caspase-3 index and Raj stage (p=0.03), but not with IgV_{H} mutation status (p=0.74) or performance status (p=0.72).

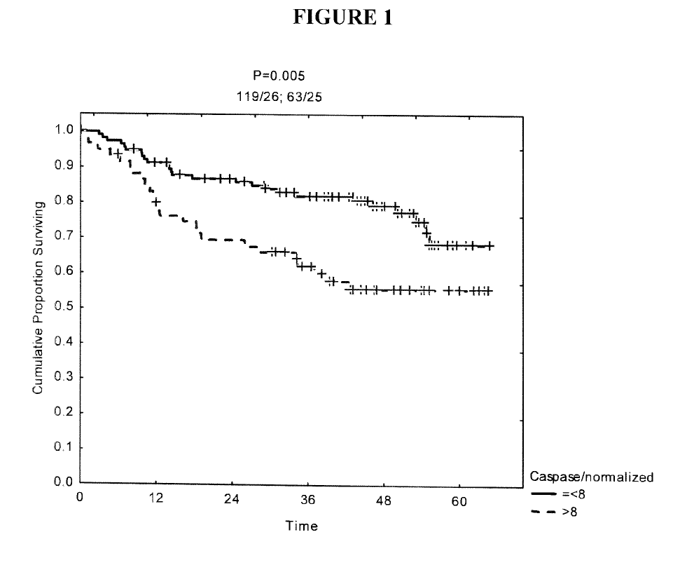

Higher caspase-3 index values (>8 pmol/min/1000 lymphocytes) was significantly associated with poor survival (p=0.005). Just over about 55% of patients survived compared to about a 78% survival rate in patients with an caspase-3 index equal to or less than 8 pmol/min/1000 lymphocytes. See FIG. 1. Multivariate statistical analysis showed that this result was not independent of B2M or IgV_{H} mutation status. Thus, a higher caspase-3 index is an indicator of more aggressive disease.

### Example 2

### Caspase-3 Activity in AML Patients

### Sample Collection

Blood was collected in EDTA-containing tubes (Becton Dickinson, N.J.) from 106 individuals with AML and 98 individuals without lymphoid malignancies. Plasma was separated from blood cells by centrifugation at 1000×g for 15 min. White blood cells were separated from RBC by lysis and centrifugation using Puregene® RBC lysis solution according to manufacturer's instructions (Qiagen, Valencia Calif.). The cell pellet was washed with phosphate-buffered saline. Both plasma and cell samples were cryopreserved at −70° C. for future use.

### Caspase-3 Assay

Caspase-3 activity was measured in each sample using an assay based on the caspase-3 peptide substrate DEVD. Five microliter of plasma was mixed with 50 μl of 2× reaction buffer with DTT. 5 μl of 1 mM of the DEVD conjugated substrate, DEVD-AMC (7-amino-4-methylcoumarin), was added to yield a final concentration of 50 μM. The mixture was incubated at 37° C. for 1 hour in a water bath. Control reactions using the caspase-3 inhibitor (DEVD-CHO) or no caspase-3 substrate were performed in parallel. Samples were read in a fluorometer with a 360-nm excitation filter and a 460-nm emission filter in 96-well plates.

### Absolute Caspase-3 Activity

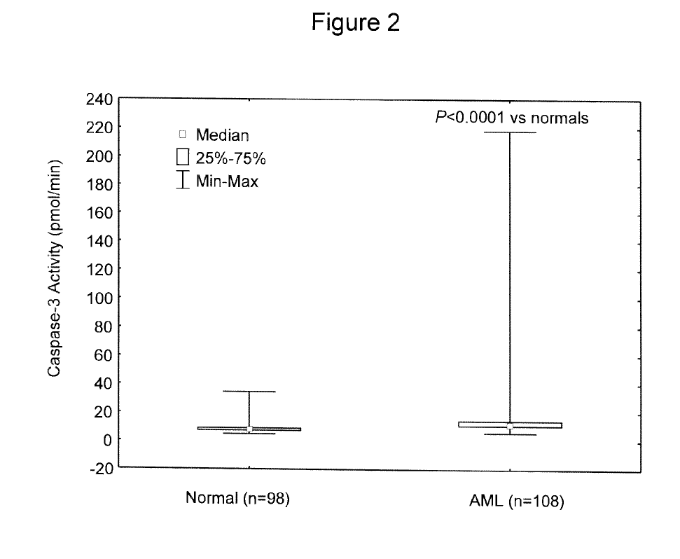

Circulating caspase-3 activity was easily detectable in the plasma of AML patients and normal controls (i.e. patients without lymphoid malignancies). The levels of absolute caspase-3 activity was significantly higher (p<0.0001) in AML patients (median=11.9 pmol/min/μl, range 5.8-218 pmol/min/μl) as compared to control patients (median 8.27 pmol/min/μl, range 4.5-34.3 pmol/min/min) as shown in FIG. 2. However, absolute levels of caspase-3 in plasma from patients with AML did not correlate with white cell count, hemoglobin, platelet count, lactate dehydrogenase, blast count, age, cytogenetic grouping, or performance status.

### Example 3

### Caspase-3 Activity and Survival of AML Patients

AML patients were divided into 2 groups based on their plasma levels of caspase-3 activity. The cut-off level was set at 14 pmol/min, a value corresponding to the upper quartile level detected in AML patients. High levels of caspase-3 activity (above 14 pmol/min) were associated with shorter survival (P=0.02) using the log-rank test as shown in FIG. 3.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

What is claimed is:

1. A method of determining a prognosis for a subject diagnosed with a leukemia comprising: i) measuring the level of caspase-3 present in a biological sample from said subject; ii) measuring the number of circulating lymphocytes in a biological sample of peripheral blood from said subject; iii) calculating a caspase-3 index by normalizing the measured caspase-3 level to the number of circulating lymphocytes; and iv) diagnosing the subject as having a poor prognosis wherein the caspase-3 index is greater than a specified cut-off value.

2. The method of claim 1, wherein the biological sample is blood serum or plasma.

3. The method of claim 1, wherein the leukemia is selected from the group consisting of: chronic lymphocytic leukemia, acute lymphoblastic leukemia, acute myelogenous leukemia, chronic myelogenous leukemia, hairy cell leukemia, T-cell prolymphocytic leukemia, large granular lymphocytic leukemia and myelodysplastic syndrome.

4. The method of claim 3, wherein the leukemia is B-cell chronic lymphocytic leukemia.

5. The method of claim 4, wherein the cut-off value is at least 8 pmol/min/100 lymphocytes.

6. The method of claim 3, wherein the leukemia is acute myelogenous leukemia.

7. The method of claim 6, wherein the cut-off value is at least 14 pmol/min/1000 lymphocytes.

8. The method of claim 1, wherein the poor prognosis is reduced survival time.

9. The method of claim 1, wherein the level of caspase-3 is measured by the amount of caspase-3 protein present in the biological sample or the amount of caspase-3 enzymatic activity present in the biological sample.

10. The method of claim 9, wherein the amount of caspase-3 enzymatic activity present in the biological sample is measured by an assay comprising a caspase-3 peptide substrate.

11. The method of claim 10, wherein the substrate comprises the peptide DEVD.

12. The method of claim 11, wherein the substrate is the peptide DEVD is linked to the fluorescent moiety 7-amino-4-methylcoumarin (AMC), 7-amino-4-trifluoromethylcoumarin (AFC) or the colorimetric moiety p-nitroanilide (pNA).
